(19) United States
(12) Patent Application Publication
(10) Pub. No.: US 2025/0177336 A1
(43) Pub. Date: Jun. 5, 2025

(54) TRANEXAMIC ACID FORMULATION AND INTRAMUSCULAR INJECTION METHOD

(71) Applicant: RX BANDZ, INC., Locust Valley, NY (US)

(72) Inventor: Stephen Harhen, Rumson, NJ (US)

(21) Appl. No.: 18/967,833

(22) Filed: Dec. 4, 2024

Related U.S. Application Data
(60) Provisional application No. 63/605,735, filed on Dec. 4, 2023.

(51) Int. Cl. A61K 31/195; A61K 9/00; A61K 31/167; A61K 47/14; A61P 7/00

(52) U.S. Cl. CPC A61K 31/195; A61K 9/0019; A61K 31/167; A61K 47/14; A61P 7/00

(57) ABSTRACT

A pharmaceutical composition is provided for treating blood loss in a patient, where the composition includes tranexamic acid at a concentration ranging from 250 mg/mL to 500 mg/mL, and a modulator in a quantity that effectively improves local muscle perfusion and reduces hypertonicity effects of the tranexamic acid upon intramuscular administration to the patient. The composition provides an effective treatment for blood loss while addressing potential side effects associated with tranexamic acid, thereby enhancing patient outcome and recovery.

### CROSS-REFERENCE TO RELATED APPLICATIONS

This application claims the benefit of U.S. Provisional Application No. 63/605,735, filed on Dec. 4, 2023. The entire disclosure of the above application is incorporated herein by reference.

### FIELD

The present technology relates to tranexamic acid formulations, and more particularly, to tranexamic acid formulations for intramuscular injection.

### INTRODUCTION

This section provides background information related to the present disclosure which is not necessarily prior art.

Tranexamic acid (TXA) is a therapeutic agent used in various medical applications, particularly in managing hemorrhage and reducing blood loss during surgery. The synthetic antifibrinolytic properties of TXA can be useful in preventing excessive bleeding by inhibiting the dissolution of blood clots. The efficacy of TXA can find use in various medical scenarios, including trauma, orthopedic surgeries, and obstetric interventions.

Controlling hemorrhage can be an acute factor in certain medical scenarios. Hemorrhage is the leading cause of preventable death on the battlefield with very high mortality rates among the victims of non-compressible hemorrhage. Non-compressible hemorrhage cannot be controlled by direct pressure and requires evacuation to a surgical capable facility. Pre-hospital treatment options are extremely limited.

Parenteral TXA products can be formulated in water for injection at a concentration of 100 mg/mL or in sodium chloride at 10 mg/mL. A significant challenge exists, however, with respect to formulations of TXA in reaching a dose of 1 g TXA. For example, TXA formulations can range from about 100 mg/ml up to about 167 mg/ml, which is about the maximum achievable soluble concentration TXA in a water solution.

Creating a concentrated TXA formulation for intramuscular injection presents therefore presents certain challenges that can lead to undesirable outcomes, primarily stemming from the hypertonic nature of the formulation. Myonecrosis, the death of muscle tissue, can occur when a hypertonic solution of TXA is injected intramuscularly, disrupting the osmotic balance and causing cellular damage within muscle fibers. This can result in tissue damage, a complicated inflammatory response, and a reduction in therapeutic uptake and bioavailability, thereby compromising the systemic antifibrinolytic effectiveness of the TXA. Factors related to pharmacokinetics and drug absorption of TXA also come into play. The rate and extent of drug absorption from the injection site can be influenced by factors such as diffusion, perfusion, and tissue binding. Injecting volumes above the physiological threshold may disrupt these processes, resulting in uneven drug distribution, delayed onset of action, and reduced therapeutic efficacy. Additionally, there is a risk of adverse reactions associated with excessive injection volumes.

There is a continuing need for formulations and administration methods that can deliver an appropriate therapeutic dose of TXA without compromising treatment efficacy. Desirably, such formulations would minimize muscle tissue damage and maximize therapeutic bioavailability while providing an effective dose of TXA in a volume suitable for intramuscular administration.

### SUMMARY

In concordance with the instant disclosure, formulations and administration methods that can deliver a therapeutic dose of tranexamic acid (TXA) for intramuscular injection have surprisingly been discovered. The present technology includes articles of manufacture, systems, and processes that relate to TXA formulations and methods for intramuscular injection that can deliver an effective dose of TXA without compromising treatment of myonecrosis, TXA uptake, inflammation, and bioavailability.

In certain embodiments, a pharmaceutical composition for treating blood loss in a patient can include TXA in a concentration from 250 mg/mL to 500 mg/mL and a vasodilator, wherein the vasodilator can be present in an amount effective to enhance local muscle perfusion and mitigate hypertonicity effects of the TXA when intramuscularly administered to the patient.

In certain embodiments, a method of treating blood loss in a patient can include intramuscularly administering to the patient an effective amount of a pharmaceutical composition comprising TXA in a concentration from 250 mg/mL to 500 mg/mL and a vasodilator in an amount effective to enhance local muscle perfusion and mitigate hypertonicity effects of the TXA.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a graphical depiction plasma concentration of TXA over time comparing intramuscular delivery of the present formulation versus intravenous administration.

FIGS. 2A-2C depict microscope images of muscle tissue samples following intramuscular injection of: 2A concentrated TXA formulation with vasodilator showing preserved muscle structure, 2B TXA without vasodilator showing extensive myonecrosis and cellular damage, and 2C saline control showing normal muscle architecture.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves intramuscular delivery of tranexamic acid (TXA) by providing formulations and administration methods that can deliver an effective dose of TXA without compromising treatment efficacy while minimizing muscle tissue damage and maximizing therapeutic bioavailability. By an “effective” amount of a drug or pharmacologically active agent is meant a nontoxic but sufficient amount of the drug or agent to provide the desired effect, e.g., reduction of bleeding. The technology can improve therapeutic outcomes by incorporating components that enhance local muscle perfusion while mitigating the hypertonicity effects of concentrated TXA when intramuscularly administered to the patient, thereby facilitating optimal drug uptake and reducing tissue damage. The technology further improves the practical administration of TXA by enabling delivery through intramuscular injection, which can provide rapid absorption and faster onset of action compared to other administration routes.

TXA is a synthetic antifibrinolytic agent that plays a role in managing hemorrhage and reducing blood loss. TXA functions by inhibiting the dissolution of blood clots, making it a valuable therapeutic agent for various medical applications including trauma, orthopedic surgeries, and obstetric interventions. TXA works as an antifibrinolytic by reversibly binding to lysine receptor sites on plasminogen and competing with fibrin for the lysine-binding sites, which militates against conversion of plasminogen into the fibrinolytic enzyme plasmin by fibrin-bound tissue plasminogen activator (tPA). As a result, TXA lowers plasmin levels, thereby preserving the framework of fibrin's matrix structure and maintaining clot strength.

The chemical structure of TXA includes important properties that affect solubility and effectiveness—TXA has pKa values of 4.3 and 10.6, and the zwitterionic form predominates at pH values close to its isoelectric point (pH˜7.3), which impacts the solubility characteristics of TXA. TXA remains functional even in severe metabolic acidosis, which is important in trauma and field care.

Formulations of the present disclosure can achieve higher concentrations through pH modification. A concentration of TXA in the formulation can range from 250 mg/mL to 500 mg/mL TXA, which can enable delivery of 500 mg TXA in a 1 mL injection volume, 1 g TXA in a 2 mL injection volume, or 2 g TXA in a 4 mL injection volume. The volumes can be specifically configured to stay within the physiological limitations of intramuscular injections. The injection volumes can be limited to no more than 5 mL per injection, as volumes exceeding this threshold can potentially lead to complications. These complications can include uneven drug distribution, tissue damage, discomfort, and risk of adverse reactions.

By formulating the solution at an acidic pH (e.g., 3.9-4.2), the solubility of TXA can be significantly increased. This can be achieved because moving away from the isoelectric point can reduce the proportion of TXA in its less soluble zwitterionic form. Through this pH modification, the formulation can achieve concentrations of 250-500 mg/mL, which can enable delivery of therapeutic doses (e.g., 1 g TXA) in volumes (e.g., 2 mL) suitable for intramuscular injection.

To this end, the formulation can include an acid. The acid can be configured to maintain an overall acidic pH of the formulation. In particular, the pH of the formulation can range from a pH of 2 to a pH of 6. More particularly, the pH of the formulation can range from a pH of 3 to a pH of 5. Most particularly, the pH of the formulation can be approximately 3.5 to 4.2. In certain embodiments, the acid can include hydrochloric acid, phosphoric acid, and combinations thereof. A skilled artisan can select other suitable acids for maintaining the pH of the formulation.

In order to maintain the pH of the formulation, a citrate buffer can be utilized. The citrate buffer can be a buffer system including citric acid and its conjugate base that helps maintain a stable pH in pharmaceutical formulations. The buffer system can function by resisting changes in pH when small amounts of acid or base are added to the solution, helping to maintain the desired pH range. Stability of the pH of the formulation can be particularly important for maintaining the high concentration of TXA in solution throughout the shelf life of the formulation.

Creating a concentrated TXA formulation for intramuscular injection presents several challenges that can lead to undesirable outcomes, primarily stemming from the hypertonic nature of the formulation. Myonecrosis, the death of muscle tissue, can occur when a hypertonic solution of TXA is injected intramuscularly, disrupting the osmotic balance and causing cellular damage within muscle fibers. This can result in tissue damage, complicated inflammatory response, and reduction in therapeutic uptake and bioavailability, compromising the systemic antifibrinolytic effectiveness of the TXA.

The formulation can include a vasomodulator to enhance local muscle perfusion while mitigating the hypertonicity effects of the concentrated TXA when intramuscularly administered to the patient. The vasomodulator can act locally to increase blood flow specifically around the injection site, minimizing the risk of systemic effects. In certain embodiments, the vasomodulator can be a vasodilator. The vasodilator, use of which is intentionally contraindicated for treatment of hemorrhaging due to its potential to exacerbate bleeding, can thereby facilitate providing an optimal amount of TXA while offsetting undesired injection volume effects. The secondary benefit to the patient can be that the increased muscle perfusion not only helps mitigate the risk of tissue damage associated with hypertonic TXA but also accelerates the rate at which the drug becomes systemically available. The inclusion of a vasomodulator, particularly a vasodilator, in a formulation containing TXA for treating bleeding presents an apparently contradictory approach. A practitioner would never administer a vasodilator alone to a bleeding patient.

TXA is specifically used to manage hemorrhage and reduce blood loss by inhibiting the dissolution of blood clots, while vasodilators work by relaxing and widening blood vessels, which could potentially exacerbate bleeding. The interaction between vasodilation and bleeding dynamics typically demands careful consideration, as increasing blood flow while managing bleeding risks requires a nuanced approach to optimize patient outcomes. However, the formulation leverages this apparent contradiction by utilizing the local effects of the vasodilator at the injection site to address the specific challenges of intramuscular TXA administration. The vasodilator acts locally to increase blood flow specifically around the injection site, minimizing the risk of systemic effects that could worsen bleeding. The vasodilator can be specifically selected for its fast-acting properties and short half-life, ensuring rapid muscle perfusion without significant delay or systemic complications. The present formulation effectively transforms what would typically be considered a contraindicated combination into a solution that enhances the therapeutic delivery of TXA while minimizing local tissue damage.

The vasodilator can include various types such as ester-type or amide-type local anesthetics. The vasodilator can be selected based on properties that enable it to act locally to increase blood flow specifically around the injection site while minimizing systemic effects. The vasodilator can be chosen for its fast-acting properties to ensure rapid muscle perfusion without significant delay. Additionally, the vasodilator can be selected to have a short half-life to limit its effects to the injection site.

The vasodilator can be included in an amount sufficient to enhance local muscle perfusion while mitigating the hypertonicity effects of the concentrated TXA when intramuscularly administered to the patient. This can help reduce inflammation and tissue damage associated with hypertonic solutions, while also providing rapid absorption, despite the counterintuitive nature of using a vasodilator in bleeding indications.

In some embodiments, the vasodilator can be lidocaine. The vasodilator can also include other suitable “-caine” family local anesthetics that provide similar vasodilatory and anesthetic properties to lidocaine. For example, the vasodilator can be an ester-type local anesthetic such as procaine, tetracaine, or benzocaine, or an amide-type local anesthetic such as bupivacaine, mepivacaine, or ropivacaine. Like lidocaine, these compounds can act locally to increase blood flow specifically around the injection site while minimizing systemic effects, and can be selected for their fast-acting properties to ensure rapid muscle perfusion without significant delay. The selection of the specific “-caine” family vasodilator can be based on properties such as onset of action, duration of effect, and local tissue perfusion characteristics that would be suitable for mitigating the hypertonicity effects of the concentrated TXA formulation.

In certain embodiments, the formulation can be provided in a delivery device configured for intramuscular administration. The delivery device can include a needle assembly, a reservoir containing the formulation, and a mechanism for delivering the formulation through the needle. The device can be configured to control the delivery rate and pressure applied during administration of the formulation. The delivery device can include features to facilitate proper insertion of the needle into tissue and subsequent delivery of the therapeutic formulation from the reservoir through the needle. Upon activation, the device can be configured to insert the needle to an appropriate depth for intramuscular administration and then deliver the formulation at a controlled rate. An example of a suitable auto-injector device for delivery of the formation is described in U.S. Provisional Patent Application Ser. No. 18/779,996 filed Jul. 22, 2024, titled FLUIDIC THROTTLE CONTROL FOR THE DELIVERY OF STANDARD AND RHEOLOGICALLY CHALLENGING THERAPIES IN NEEDLE BASED DRUG DELIVERY SYSTEMS, the entire disclosure of which is incorporated herein by reference.

The present disclosure includes methods for treating conditions associated with blood loss in a patient. These conditions can include excessive bleeding, hemorrhage, or other related disorders where TXA administration would be beneficial. The methods can include intramuscular administration of an effective amount of the formulation described herein. The methods can include treating conditions such as trauma, orthopedic surgeries, and obstetric interventions where TXA has demonstrated efficacy in preventing excessive bleeding by inhibiting the dissolution of blood clots. The formulation can be particularly useful in non-compressible hemorrhage situations where other measures may falter and in cases of primary and secondary fibrinolysis. The methods can include administering the formulation using a delivery device configured for intramuscular administration, such as a prefilled syringe or auto-injector. The delivery device can be configured to insert the needle to an appropriate depth for intramuscular administration and then deliver the formulation at a controlled rate to minimize tissue damage while ensuring proper drug delivery.

The following example applications illustrate various clinical scenarios where the concentrated TXA formulation with a vasomodulator can be particularly beneficial. These examples demonstrate how the formulation's unique properties—including its concentrated form suitable for intramuscular delivery, incorporation of a vasodilator to enhance local perfusion, and ability to be rapidly administered—address key challenges in treating blood loss across different medical situations. The examples highlight applications ranging from emergency pre-hospital care to planned surgical interventions, showcasing the versatility and utility of the formulation in situations where rapid administration of TXA is important for patient outcomes.

Application 1: Trauma and Hemorrhage in Pre-Hospital Settings

In emergency trauma situations where a patient is experiencing significant blood loss, rapid administration of TXA is important. The formulation can be administered via intramuscular injection by first responders or emergency medical personnel in pre-hospital settings where IV access may be challenging or delayed.

The concentrated formulation allows for delivery of an effective dose through intramuscular administration, which is particularly important given that TXA is most effective when given in the first 15 minutes of injury. IV administration can take 20+ minutes when accounting for emergency response time and IV setup. The vasodilator component helps ensure rapid absorption and bioavailability of the TXA, even in patients experiencing shock-induced reduction in blood pressure and muscle perfusion. This helps maintain the time-sensitive therapeutic window while avoiding tissue damage from the concentrated formulation.

Application 2: Surgical Applications

During surgical procedures where bleeding risk is elevated, such as orthopedic surgeries, the formulation can be administered intramuscularly as a prophylactic measure. The concentrated formula allows for delivery of therapeutic levels of TXA while maintaining a volume suitable for intramuscular injection. The local vasodilatory effects help distribute the medication rapidly through the muscle tissue, while the antifibrinolytic properties of TXA help prevent excessive bleeding during the procedure. The formulation's design helps avoid the logistical challenges of IV administration while still providing effective hemorrhage prevention. The controlled local action of the vasodilator component helps maintain the balance between enhancing TXA absorption and managing bleeding risks, particularly important in surgical contexts where bleeding control is important.

Application 3: Treatment of Secondary Fibrinolysis

In patients with medication-induced secondary fibrinolysis or predisposition to excessive bleeding, the formulation can provide rapid intervention to stabilize clot formation. The intramuscular administration route allows for quick deployment of therapy when needed. The concentrated formula enables delivery of therapeutic doses in volumes below 4 mL, making it suitable for intramuscular administration while avoiding the tissue damage risks associated with larger volume injections. This is particularly important for patients who may require repeated treatments. The vasodilator component helps ensure consistent drug absorption and bioavailability, while the acidic pH of the formulation maintains the high concentration of TXA needed for therapeutic effect. This combination helps provide reliable treatment for ongoing fibrinolysis management.

### EXAMPLES

Example embodiments of the present technology are provided with reference to the several figures enclosed herewith.

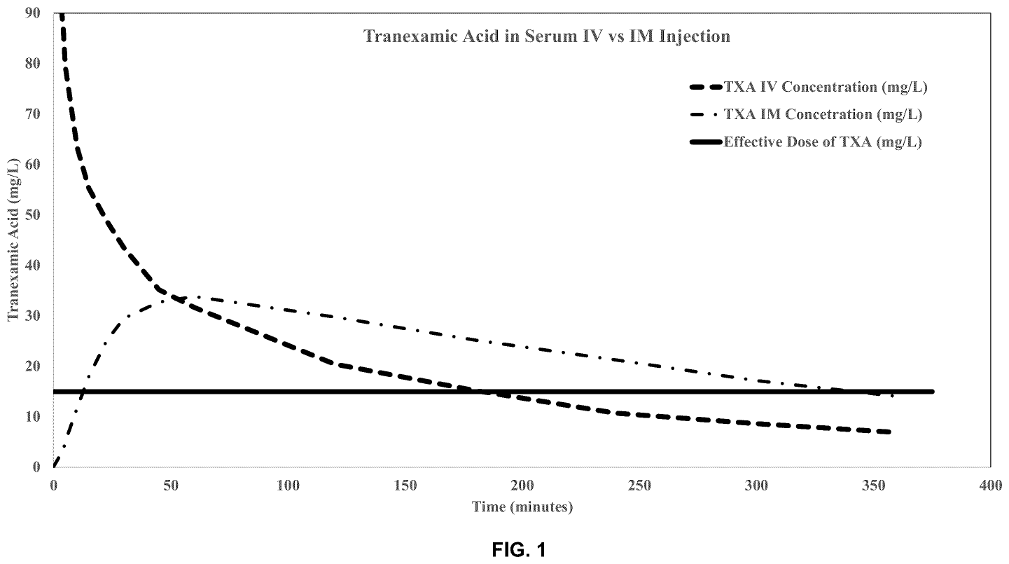

FIG. 1 depicts the pharmacokinetic profile comparing intramuscular (IM) versus intravenous (IV) administration of TXA. The graph demonstrates that while IV administration provides an initial spike in TXA concentration, the IM formulation maintains therapeutic levels over a longer duration. The IM formulation reaches effective therapeutic concentrations within 15 minutes of administration, which is important given that TXA is most effective when given in the first 15 minutes after injury. This rapid achievement of therapeutic levels addresses the time-sensitive nature of TXA administration while avoiding the delays associated with IV access and setup in emergency situations.

The sustained therapeutic concentrations achieved through IM delivery can be attributed to the inclusion of a vasodilator helps ensure consistent drug absorption and bioavailability by enhancing local muscle perfusion, while the acidic pH maintains the high concentration of TXA needed for therapeutic effect. The combination enables reliable maintenance of therapeutic levels over an extended period compared to IV administration. The extended duration of therapeutic concentrations with IM delivery is particularly advantageous in situations where ongoing antifibrinolytic activity is needed, such as trauma cases or surgical interventions. The pharmacokinetic profile demonstrates how the formulation successfully addresses both the need for rapid onset and sustained therapeutic effect.

The administration of concentrated TXA formulations via intramuscular injection presents significant challenges due to the hypertonic nature of the solution. Hypertonic solutions can cause cellular damage, myonecrosis, and inflammation when injected into muscle tissue. When a hypertonic solution is injected intramuscularly, it can disrupt the osmotic balance between extracellular fluid and muscle cells, potentially leading to cellular shrinkage, water loss, and tissue death.

The addition of a vasodilator to concentrated TXA formulations represents a novel approach to mitigating these tissue damage effects while maintaining therapeutic efficacy. By increasing local muscle perfusion, the vasodilator may help dilute the hypertonic solution before significant cellular damage can occur, while also potentially enhancing the bioavailability of the TXA.

To evaluate the protective effects of including a vasodilator in concentrated TXA formulations, a study can be conducted to evaluate local tissue response following intramuscular administration of three different formulations: (1) the concentrated TXA formulation with vasodilator, (2) concentrated TXA without vasodilator, and (3) saline control. The study would examine the effects of hypertonicity and the protective benefits of including a vasodilator.

Test subjects would receive standardized 2 mL intramuscular injections in the thigh muscle. Tissue samples would be collected at predetermined timepoints (e.g., 15 minutes, 1 hour, 4 hours, and 24 hours post-injection) to assess markers of cellular damage, inflammation, and tissue integrity. Key endpoints would include measurements of myonecrosis, cellular shrinkage, water loss, and inflammatory markers.

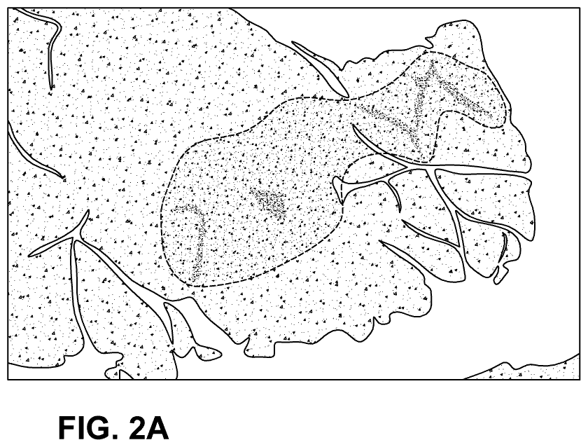

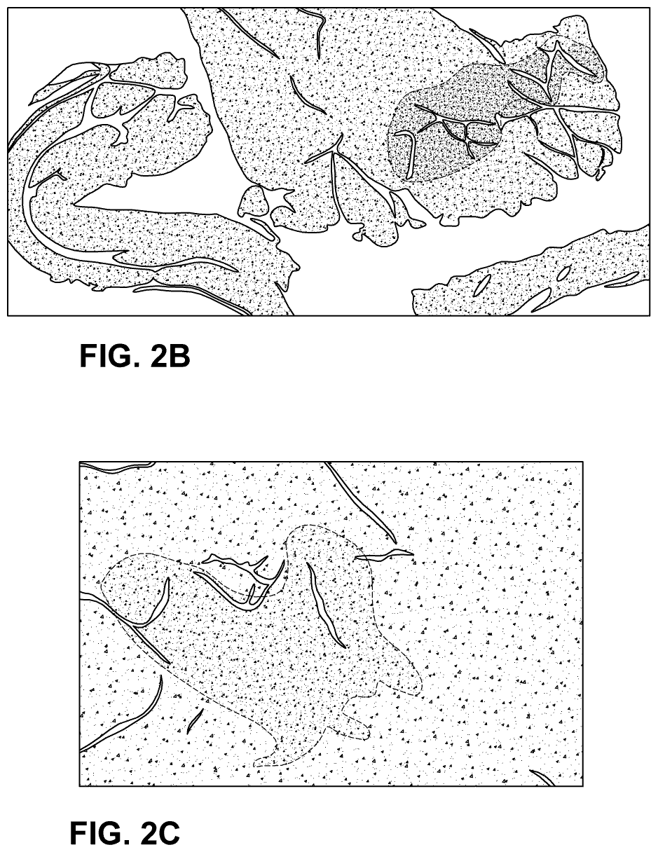

The results of histological analysis are shown in FIGS. 2A-2C, which depict microscope slides of muscle tissue samples.

FIG. 2A depicts microscope images of tissue samples following intramuscular injection of the concentrated TXA formulation with vasodilator, showing preserved muscle fiber structure, minimal cellular infiltration, and maintenance of normal tissue architecture. FIG. 2B shows microscope images revealing extensive myonecrosis, cellular damage, and inflammatory infiltration in tissue samples from TXA without vasodilator. FIG. 2C provides microscope images of baseline tissue response from saline control injections, demonstrating normal muscle architecture with minimal disruption.

Histological examination of the tissue samples would evaluate the extent of muscle fiber damage, cellular infiltration, and tissue architecture. The degree of myonecrosis would be quantified through measurement of muscle enzyme release (e.g., creatine kinase) into circulation. Local blood flow measurements would assess the effectiveness of the vasodilator in maintaining muscle perfusion. It is expected that the TXA formulation containing the vasodilator would show significantly reduced tissue damage compared to TXA without vasodilator, while maintaining therapeutic efficacy.

The saline control group would establish baseline tissue response to intramuscular injection, while the TXA-only group would demonstrate the tissue damage typically associated with hypertonic solutions. The addition of the vasodilator would be expected to increase muscle perfusion and dilute the hypertonic TXA solution before cellular shrinkage, water loss, or resultant death can occur.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A pharmaceutical composition for treating blood loss in a patient, comprising:
tranexamic acid in a concentration from 250 mg/mL to 500 mg/mL; and
a vasomodulator in an amount effective to enhance local muscle perfusion and mitigate hypertonicity effects of the tranexamic acid when intramuscularly administered to the patient.

2. The pharmaceutical composition of claim 1, wherein the vasomodulator is a vasodilator.

3. The pharmaceutical composition of claim 2, wherein the vasodilator is an ester-type or amide-type local anesthetic.

4. The pharmaceutical composition of claim 2, wherein the vasodilator is selected from the group consisting of lidocaine, procaine, tetracaine, benzocaine, bupivacaine, mepivacaine, and ropivacaine.

5. The pharmaceutical composition of claim 4, wherein the vasodilator is lidocaine.

6. The pharmaceutical composition of claim 1, further comprising an acid configured to provide a pH between 3.5 and 4.2.

7. The pharmaceutical composition of claim 5, wherein the acid comprises hydrochloric acid, phosphoric acid, or combinations thereof.

8. The pharmaceutical composition of claim 1, further comprising a citrate buffer.

9. The pharmaceutical composition of claim 1, wherein the composition is configured for intramuscular injection in a volume below 4 mL.

10. The pharmaceutical composition of claim 1, wherein the tranexamic acid is present in an amount of 1 gram in a 2 mL volume.

11. A method of treating blood loss in a patient, comprising:
intramuscularly administering to the patient an effective amount of a pharmaceutical composition including:
tranexamic acid in a concentration from 250 mg/mL to 500 mg/mL; and
a vasodilator in an amount effective to enhance local muscle perfusion and mitigate hypertonicity effects of the tranexamic acid.

12. The method of claim 11, wherein the vasomodulator is a vasodilator.

13. The method of claim 12, wherein the vasodilator is an ester-type or amide-type local anesthetic.

14. The method of claim 12, wherein the vasodilator is selected from the group consisting of lidocaine, procaine, tetracaine, benzocaine, bupivacaine, mepivacaine, and ropivacaine.

15. The method of claim 12, wherein the vasodilator is lidocaine.

16. The method of claim 11, wherein the pharmaceutical composition further comprises an acid configured to maintain the composition at a pH between 3.5 and 4.2.

17. The method of claim 11, wherein intramuscularly administering comprises delivering the pharmaceutical composition in a volume below 4 mL.

18. The method of claim 11, wherein the pharmaceutical composition is administered within 15 minutes of the patient experiencing trauma or injury.

19. The method of claim 11, wherein intramuscularly administering includes using an auto-injector or prefilled syringe.

20. The method of claim 11, wherein the tranexamic acid is present in an amount of 1 gram in a 2 mL volume.
